(19) United States
(12) Patent Application Publication
(10) Pub. No.: US 2016/0161369 A1
(43) Pub. Date: Jun. 9, 2016

(54) METHOD FOR ISOLATING QUANTITY ERRORS OF A FUEL AMOUNT AND AN AIR AMOUNT DELIVERED TO AT LEAST CYLINDER OF AN INTERNAL COMBUSION ENGINE

(71) Applicant: ROBERT BOSCH GMBH, Stuttgart (DE)

(72) Inventors: Uwe Mueller, Cleebronn (DE); Alexandra Fuchsbauer, Stuttgart (DE); Armin Hassdenteufel, Sachsenheim-Ochsenbach (DE); Guido Porten, Wiernsheim (DE); Matthias Walz, Wiernsheim (DE)

(21) Appl. No.: 14/903,978

(22) PCT Filed: May 20, 2014
(86) PCT No.: PCT/EP2014/060293

§ 371 Date: Jan. 8, 2016

(30) Foreign Application Priority Data
Jul. 9, 2013 (DE) 10 2013 213 405.3

(51) Int. Cl. G01M 15/08; G01N 33/22; G01N 33/00

(52) U.S. Cl. CPC G01M 15/08; G01N 33/0009; G01N 33/225

(57) ABSTRACT

A method for determining quantitative errors in a fuel quantity and air quantity delivered to at least one cylinder of an internal combustion engine, in which in a first phase a cylinder equalization of the internal combustion engine is accomplished, and an error in the fuel quantity delivered to the at least one cylinder is determined therefrom. In a second phase the internal combustion engine is operated with a stoichiometric ratio of air quantity and fuel quantity, a feature of the at least one cylinder correlating with an indicated mean pressure is sensed, and an error in the air quantity delivered to the at least one cylinder is determined from the feature correlating with the indicated mean pressure.

### FIELD OF THE INVENTION

The present invention relates to a method for determining quantitative errors in a fuel quantity and air quantity delivered to at least one cylinder of an internal combustion engine.

### BACKGROUND INFORMATION

In internal combustion engines, especially gasoline engines, a fresh-air quantity or air quantity and a fuel quantity are delivered to the individual cylinders of the internal combustion engine during a combustion cycle. The air quantity is sensed instrumentally. The fuel quantity is metered by way of a suitable pilot control system as a function of the air quantity so that stoichiometric combustion is established. An error that may be present in the air quantity and/or fuel quantity is detected by the lambda probe and compensated for by lambda regulation of the fuel quantity. This intervention by the lambda regulation system can be adapted and can be used for better pilot control, i.e. in the future, a more accurate fuel quantity is generated for the air quantity that is ascertained. The adaptation value and the lambda regulation intervention do not, however, provide any conclusion as to whether the air quantity was incorrectly sensed or the fuel quantity was incorrectly metered. The engine is thus operated with inaccurate pilot control, which can result in an environmental impact. Furthermore, in the event of an error, i.e. if the lambda regulation intervention exceeds a specific threshold value, it is impossible to detect whether the error derives from the air system or from the fuel system.

It is therefore desirable to furnish a capability for detecting errors in a delivered air quantity and in a delivered fuel quantity in an internal combustion engine in a simple and economical manner and separately from one another.

### SUMMARY OF THE INVENTION

The present invention proposes a method, having the features described herein, for determining quantitative errors in a fuel quantity and air quantity delivered to at least one cylinder of an internal combustion engine. Advantageous embodiments are the subject matter of the further descriptions herein and of the description that follows.

The method according to the present invention is subdivided into two phases. In a first phase a detection of an error in the delivered fuel quantity occurs, and in a second phase a detection of an error in the delivered air quantity occurs. Respective errors in the delivered fuel quantity and in the delivered air quantity of at least one of the cylinders or of several, in particular all, cylinders of the internal combustion engine, can thereby be detected. The description below refers to a usefully selected number of cylinders of the internal combustion engine.

The method according to the present invention allows errors in the delivered air quantity and in the delivered fuel quantity to be determined separately from one another. Malfunctioning or roughness of the internal combustion engine can thus be attributed unequivocally to errors in air delivery, for example in an air intake system, or in fuel delivery, for example in a fuel injection system. This does not require complex or cost-intensive additional sensor equipment. The components present in any case in the internal combustion engine can be used. The method according to the present invention can furthermore be carried out during ordinary operation of the internal combustion engine.

In the first phase a cylinder equalization of the internal combustion engine is accomplished, all cylinders of the internal combustion engine being equalized in terms of an engine variable. For example, injection valves of the cylinders can be equalized in terms of the delivered fuel quantity. Reference is made, for example, to DE 10 2007 020 964 A1 for a detailed description of a cylinder equalization of an internal combustion engine.

In the second phase, the internal combustion engine is operated in a stoichiometric mode, i.e. a “lambda=1” mode. Here the required fuel quantity is calculated from the delivered air quantity in consideration of the rotation speed, and optionally corrected via lambda regulation. A stoichiometric fuel-air mixture is established, where lambda=1.

A feature of the at least one cylinder correlating with an (indicated) mean pressure (pmi) is sensed. The indicated mean pressure is an indicator of an amount of work performed by the respective cylinder, with respect to a corresponding piston displacement. From this feature correlating with the indicated mean pressure, an error in the delivered air quantity of the pertinent cylinder is determined. The determination of the indicated mean pressure may be accomplished at a defined operating point of the internal combustion engine, at lambda=1 and with the lambda regulation system at equilibrium.

In the second phase a first value for a torque of the internal combustion engine may be determined from the feature correlating with the indicated mean pressure. A second value for the torque of the internal combustion engine is determined by way of a measured value of the delivered air quantity. This second value is determined in particular by an engine control system, in particular by a control unit of the internal combustion engine. This second value is usually determined in any case, and can be used for the method according to the present invention. The internal combustion engine operates in the stoichiometric mode in air-priority fashion, i.e. the resulting torque of the internal combustion engine depends on the air quantity actually present in the cylinder. The first value and second value for the torque of the internal combustion engine are therefore compared with one another. If a difference between the first value and second value for the torque reaches a threshold value, this indicates an error in the delivered air quantity. The error can be an error in the sensing of the air quantity and/or incorrect air, i.e. delivery of too much or too little air.

Both global methods (i.e. over all cylinders) and individual-cylinder methods for determining torque are possible in practice. A global or individual-cylinder determination of an air error results as a function thereof.

In an advantageous embodiment of the invention, in the second phase a value of the air quantity error and a value of the fuel quantity error are determined. Thus not only is a determination made that an error exists in the delivered air quantity or the delivered fuel quantity, but the errors are also quantified and a value of that error in the delivered fuel quantity or air quantity, hereinafter referred to as a value of the air quantity error or of the fuel quantity error, is determined.

From the first value for the torque, which is determined from the feature correlating with the indicated mean pressure, a theoretical value for the air quantity is determined. This theoretical value for the air quantity can be determined in particular by way of a characteristics diagram that describes a relationship between torque and air quantity. The value of the air quantity error is determined in particular as a difference between the theoretical value for the air quantity and the measured value of the delivered fuel quantity. As already explained, the resulting torque of the internal combustion engine in stoichiometric mode depends on the air quantity actually present in the cylinder. In stoichiometric mode, errors in the delivered fuel quantity thus have no influence on the resulting torque of the internal combustion engine.

As mentioned at the outset, if an error in the delivered air quantity and/or fuel quantity is determined, an intervention by a lambda regulation system is adapted by an amount equal to an adaptation value. In particular, the value of the fuel quantity error is determined by compensating or decreasing this adaptation value by an amount equal to the determined value of the air quantity error. The value of the fuel quantity error here is independent of an operating point of the internal combustion engine. If the value of the fuel quantity error is determined in the second phase at the defined steady-state operating point of the internal combustion engine, the value of the fuel quantity error can be used for all other operating points of the internal combustion engine. The value of the air quantity error may thus be determined, by way of the value of the fuel quantity error, even when the internal combustion engine is being operated outside the second phase at any appropriate operating point. The value of the air quantity error is obtained in each case from a current value of the adaptation value of the lambda regulation, compensated or decreased by an amount equal to the value of the fuel quantity error.

The error in the delivered air quantity and the error in the delivered fuel quantity may be corrected respectively by way of the value determined for the air quantity error and the value determined for the fuel quantity error. The respective errors are thus not only detected but also corrected. In particular, by way of the value determined for the air quantity error and the value determined for the fuel quantity error, an air quantity and a fuel quantity are respectively corrected by a pilot control system of the internal combustion engine. This makes it possible to deliver a correct air quantity and fuel quantity to the internal combustion engine. Unnecessarily high fuel consumption by the internal combustion engine is thus prevented, and environmental impacts are reduced.

An error in the delivered air quantity can be used to improve or adapt the air quantity measurement, so that an instrumental ascertainment of the air quantity, or one based on a model (e.g. intake manifold pressure model), is adapted in such a way that the new ascertained air quantity corresponds to the air quantity actually delivered.

The torque can be a total torque of all cylinders of the internal combustion engine, or torque contributions of individual cylinders to the total torque.

The feature correlating with the indicated mean pressure may be the indicated mean pressure itself. A combustion chamber pressure sensor may be present in the respective cylinder for determination of the indicated mean pressure of a cylinder. If a combustion chamber pressure sensor is not present in the cylinders, the feature correlating with the indicated mean pressure can be a feature, based on a rotation speed, for the mechanical work of the at least one cylinder of the internal combustion engine (mechanical work feature, MWF). The MWF is a feature, determinable with little calculation outlay, for the work delivered as a result of combustion. A combustion chamber pressure sensor is not needed for determination of the MWF. The MWF can be calculated, for example, from an energy balance of a crankshaft of the internal combustion engine in a defined applicable angle range. For example, measured inter-tooth times of an encoder wheel can be used for this. Reference is made, for example, to Application DE 10 2012 203 652 for a detailed description of the properties and determination of MWF and pmi.

Advantageously, the internal combustion engine is fueled in a leaned mode for cylinder equalization in the first phase. Here all the cylinders are leaned out simultaneously and a smoothness signal is determined. An equalization of the cylinders is accomplished based on the ascertained smoothness signal. This is possible because in lean mode, the torque delivered by the cylinders (which influences the smoothness signal) correlates with the fuel quantity. If the cylinder equalization intervention exceeds a threshold value for a cylinder, an error therefore exists in the fuel path for the cylinder in question.

In particular, the internal combustion engine is fueled in a lean mode. Because, in lean mode, torque is proportional to the delivered fuel quantity, measurement tolerances of components, for example injection valves, can be compensated for to a high degree. For example, if combustion chamber pressure sensors are installed in the internal combustion engine, for cylinder equalization the indicated mean pressures of the individual cylinders can be equalized. An error in the delivered fuel quantity is therefore advantageously determined by way of this proportionality between the torque of the internal combustion engine and the delivered fuel quantity. In particular, the torque itself is determined as an individual-cylinder feature.

In an exemplary embodiment of the invention, a non-torque-effective post-injection takes place in the first phase. Here fuel is injected into a combustion chamber of the cylinder or cylinders in torque-neutral fashion with regard to evaluation of the cylinder equalization. The post-injection is calculated in such a way that exhaust gas of the combustion cycle of the internal combustion engine in lean mode corresponds substantially to a stoichiometric air-fuel mixture, i.e. so that an exhaust-gas-neutral summed lambda value (lambda=1) results. A procedure of this kind has the advantage that cylinder equalization can be effected in exhaust-gas-neutral fashion even in a homogeneous operating mode of the internal combustion engine.

The point in time of the post-injection is usefully calculated exactly. If the post-injection occurs too early, the post-injection also generates an appreciable torque contribution that becomes perceptible in the evaluation of the smoothness signal. If the post-injection occurs too late, complete combustion of the post-injected fuel is not possible. The post-injection is therefore accomplished in such a way that any torque contribution of the post-injection which may be present is negligible in terms of evaluation of the cylinder equalization.

A calculation unit according to the present invention, for example a control unit of a motor vehicle, is configured to carry out, in particular by programmed execution, a method according to the present invention.

Implementation of the method in the form of software is also advantageous, since it entails particularly low costs, in particular if an executing control unit is also used for further tasks and is therefore present in any case. Suitable data media for furnishing the computer program are, in particular, diskettes, hard drives, flash memories, EEPROMs, CD-ROMs, DVDs, and others. Downloading of a program via computer networks (internet, intranet, etc.) is also possible.

Further advantages and embodiments of the invention are evident from the description and the appended drawings.

It is understood that the features recited above and those yet to be explained below are usable not only in the respective combination indicated, but also in other combinations or in isolation, without departing from the scope of the present invention.

The invention is schematically depicted in the drawings on the basis of exemplifying embodiments and will be described below in detail with reference to the drawings.

### BRIEF DESCRIPTION OF THE DRAWINGS

FIG. 1 schematically shows a portion of an internal combustion engine that is configured to carry out an embodiment of a method according to the present invention.

FIG. 2 schematically shows, as a block diagram, an embodiment of a method according to the present invention.

FIG. 3 schematically shows, as a block diagram, a further embodiment of a method according to the present invention.

### DETAILED DESCRIPTION

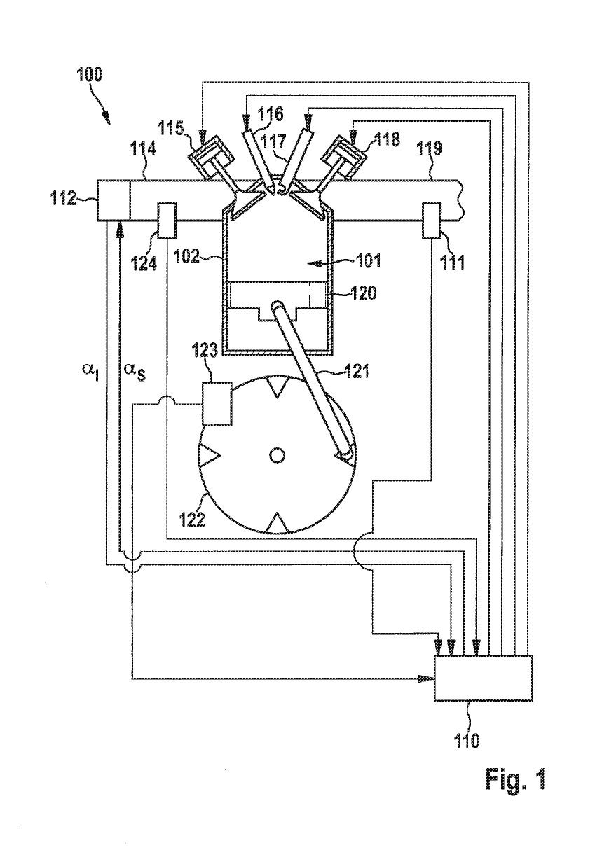

A portion of an internal combustion engine, for example of an Otto cycle engine or diesel engine, is depicted schematically in FIG. 1 and labeled 100. Internal combustion engine 100 has a calculation unit, embodied as control unit 110, that is configured to carry out an embodiment of a method according to the present invention. Internal combustion engine 100 furthermore encompasses multiple cylinders, although for the sake of clarity only a first cylinder 102 is depicted. First cylinder 102 of internal combustion engine 100 encompasses a combustion chamber 101 to which a quantity of fresh air is delivered via a throttle valve 112 and an intake duct 114 disposed between throttle valve 112 and an intake valve 115. An air mass sensor 124, which senses this fresh air as a delivered air mass or air quantity, is disposed in the intake duct. Air mass sensor 124 conveys the measured values for the delivered air quantity to control unit 110.

In addition, a fuel quantity is injected or delivered into combustion chamber 101 through an injection valve 116. Injection valve 116 is disposed on combustion chamber 101, for example, in such a way that fuel is injected directly into combustion chamber 101. The fuel quantity can be metered in as a function of the air quantity. A fuel-air mixture thereby produced is combusted in combustion chamber 101. In the case of an Otto cycle engine, internal combustion engine 100 usually encompasses for this purpose a spark plug 117, which is likewise disposed on combustion chamber 101.

An exhaust gas resulting from combustion is guided through an exhaust gas valve 118 disposed on combustion chamber 101, through an exhaust duct 119, and past a lambda sensor 111. Control unit 110 receives in this context, from lambda sensor 111, a lambda signal that reproduces the oxygen concentration in the exhaust gas of the internal combustion engine.

A thermal energy resulting from combustion of the fuel-air mixture in combustion chamber 101 is transferred at least in part, via a piston 120 via a connecting rod 121, to a crankshaft 122. A rotary motion is thereby imparted to crankshaft 122. The rotary motion of crankshaft 122, in particular a rotation speed of internal combustion engine 100, is determined by a rotation speed sensor 123. Rotation speed sensor 123 conveys to control unit 110 the rotation speed that is determined.

Control unit 110 furthermore receives a current throttle valve angle α_{I} from throttle valve 112 as an actual value, and conveys to throttle valve 112 a target value α_{S} for the throttle valve angle. Control unit 110 furthermore ascertains control application signals for intake valve 115, exhaust valve 118, injection valve 116, and spark plug 117. These control application variables are ascertained, for example, from the rotation speed, the actual value α_{I} of the throttle valve angle, and the aspirated air quantity.

Individual-cylinder filling differences can result in different torque contributions and thus in rough running of internal combustion engine 100. These individual-cylinder torque contributions can be brought about by errors either in the aspirated or delivered air quantity or in the injected fuel quantity. On the one hand, an air quantity actually introduced into cylinder 102 can deviate, for example because of contamination or irregular distribution in intake duct 114, from the measured total air quantity divided by the number of cylinders. On the other hand, the fuel quantity actually injected by injection valve 116 can deviate from a specific target value as a result of tolerances of injection valve 116.

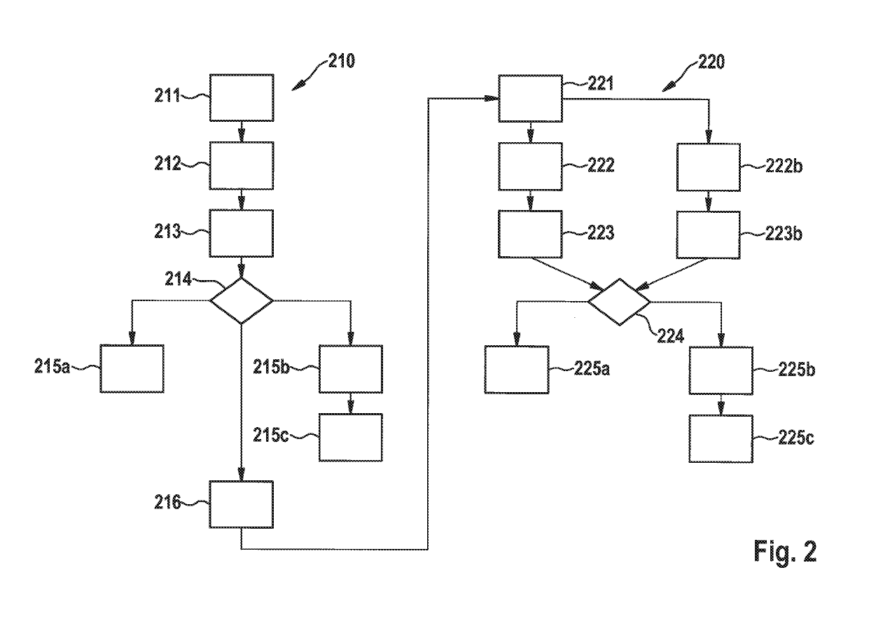

In order to distinguish as to whether an error in the air quantity or an error in the injected fuel quantity exists, control unit 110 carries out an embodiment of a method according to the present invention which is depicted schematically in FIG. 2 in the form of a block diagram.

In a first phase 210, an equalization of the cylinders of the internal combustion engine is carried out. In this specific example, the equalization is accomplished in such a way that in a first step 211 internal combustion engine 100 is fueled in a leaned mode, an excess of air being generated in combustion chamber 101.

In step 212, firstly a smoothness signal is determined. Different torque contributions by the individual cylinders result in different accelerations of crankshaft 122, which are expressed as different segment times or inter-tooth times. “Segment times” describe time periods required by the crankshaft to traverse a specific angle range. The torque contribution of first cylinder 102 occurs, for example, in an angle range between 180° and 360° crankshaft angle (CA). The segment time during which the torque contribution of first cylinder 102 occurs is, for example, the time period required by the crankshaft to traverse the angle range from 180° to 360° crankshaft angle. A smoothness signal is ascertained from a comparison among the segment times of the individual cylinders. For example, the individual-cylinder segment time is compared with an average of all segment times. The deviation of the individual-cylinder segment time from the average corresponds to the roughness.

The smoothness signal is evaluated in step 213, and the fuel quantity of the individual cylinders is equalized on the basis thereof, for example by adjusting the segment times.

Step 214 checks whether the interventions in the context of equalization of the cylinders are greater than a threshold value. If this is not the case, no error exists (step 215a). If this is the case, however, a error in the injected fuel quantity (injection valve component error) can be inferred (step 215b).

In order for exhaust-gas-neutral combustion to take place, and for an individual-cylinder lambda value of 1 to be maintained, even during operation of internal combustion engine 100 in a leaned mode, a post-injection takes place in step 216. The post-injection usefully occurs at a point in time at which combustion of the post-injected fuel quantity no longer supplies a substantial torque contribution.

Second phase 220 of the embodiment of the method according to the present invention may take place at a defined steady-state operating point of internal combustion engine 100. In the second phase, internal combustion engine 100 is fueled in a stoichiometric lambda=1 mode (step 221).

If a combustion chamber pressure sensor is present in combustion chamber 101, then in step 222 an indicated mean pressure is determined as a feature of cylinder 102 correlating with the indicated mean pressure. If a combustion chamber pressure sensor is not present in combustion chamber 101, then in step 222 a mechanical work feature (MWF), based on a rotation speed, of cylinder 102 is determined as a feature correlating with the indicated mean pressure. The MWF is determined in control unit 110 from inter-tooth times of an encoder wheel (not depicted in FIG. 1).

From this feature correlating with the indicated mean pressure of the internal combustion engine, in step 223 a first value for the torque of internal combustion engine 100 is determined in control unit 110.

In the stoichiometric lambda=1 mode of internal combustion engine 100, in step 222b the injected fuel quantity is pilot-controlled as a function of the air quantity ascertained by air mass sensor 124. The injected fuel quantity is corrected, by way of a lambda regulation, in such a way that combustion occurs with a lambda value of 1. The fuel injection occurs at a point in time that is favorable for combustion and torque generation. As a function of the said air quantity ascertained by air mass sensor 124, in step 223b a second value for the torque of the internal combustion engine is determined. This second value for the torque of the internal combustion engine is usually determined in any case in control unit 110, and can be used for the second phase of the method according to the present invention.

In step 224, the first value and second value for the torque of internal combustion engine 100 are compared with one another. In particular, the two values for the torque of internal combustion engine 100 are subtracted from one another. If the absolute value of this difference is below an appropriately selected limit value, no error exists (step 225a). If the absolute value of this difference exceeds the limit value, this indicates a error in the aspirated air quantity of cylinder 102 (step 225b).

In the event of an error in the injected fuel quantity (already ascertained in phase 1) or in the aspirated fuel quantity, an “incorrect delivered fuel quantity” status or “incorrect delivered air quantity” status (step 215c or 225c, respectively) is saved in a memory in control unit 110. Alternatively or additionally, the corresponding “incorrect delivered fuel quantity” or “incorrect delivered air quantity” information item can also be outputted to a driver of the motor vehicle.

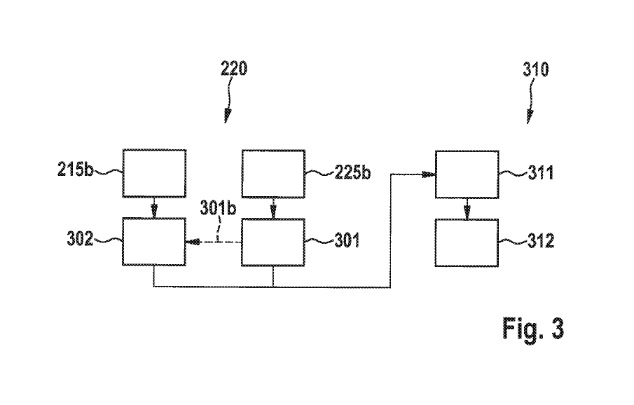

Alternatively or additionally to saving of the status or information output to the driver respectively in step 215c or step 225c, a correction 310 of the errors can also be carried out, in accordance with a further embodiment of the invention that is depicted schematically in FIG. 3 in the form of a block diagram.

If an error in the aspirated air quantity of cylinder 102 is determined in accordance with step 225b, then in second phase 220, firstly a value for this air quantity error is determined in step 301. For this, a theoretical value for the air quantity is determined by way of the first value, determined in step 223, for the torque of internal combustion engine 100. This theoretical value is determined in control unit 110, for example by way of a characteristics diagram that describes a relationship between torque and air quantity. The value of the air quantity error is determined as a difference between this theoretical value of the air quantity and the air quantity ascertained by air mass sensor 124.

If an error in the injected fuel quantity is determined in accordance with step 215b, then in second phase 220 a value for this fuel quantity error is determined in step 302. This value for the fuel quantity error is determined from an adaptation value for an adaptation of a regulation of lambda sensor 111. If an error in the aspirated air quantity of cylinder 102 is also determined in accordance with step 225b, the value for the fuel quantity error is obtained as a difference between the adaptation value and the value, determined in step 301, of the air quantity error (indicated by reference character (301b). If, according to step 225a, no error exists in the aspirated air quantity of cylinder 102, the value for the fuel quantity error is obtained as this adaptation value.

By way of these determined values of the air quantity error and fuel quantity error, a correction 310 of the errors in the aspirated air quantity and injected fuel quantity is carried out. The specific example in which an error exists both in the aspirated air quantity and in the injected fuel quantity will be considered below.

Internal combustion engine 100 is operated at an arbitrary appropriate operating point. In step 311, a current value of the air quantity error for this arbitrary appropriate operating point is determined in control unit 110. Because the value of the fuel quantity error, determined in step 302, is independent of the operating point of internal combustion engine 100, this value is also valid for this arbitrary appropriate operating point. In step 311, the current value of the air quantity error is determined as a difference between the adaptation value and the value, determined in step 302, of the fuel quantity error.

In step 312, control application signals of control unit 110 to intake valve 115 and to injection valve 116, and the target value α_{S} for the throttle valve angle of throttle valve 112, are corrected based on the value, determined in step 302, of the fuel quantity error and on the current value, determined in step 311, of the air quantity error. This ensures that the air quantity aspirated by intake valve 115 and the fuel quantity injected by injection valve 116 are correct and error-free. The errors in the aspirated fuel quantity and in the injected fuel quantity can thus be corrected.

1-15. (canceled)

16. A method for determining a quantitative error in a fuel quantity and air quantity delivered to at least one cylinder of an internal combustion engine, the method comprising:
providing, in a first phase, a cylinder equalization with regard to the fuel quantity delivered to the internal combustion engine;
determining, in the first phase, an error in the fuel quantity delivered to the at least one cylinder therefrom;
operating, in a second phase, the internal combustion engine with a stoichiometric ratio of air quantity and fuel quantity;
sensing, in the second phase, a feature of the at least one cylinder correlating with an indicated mean pressure;
determining, in the second phase, an error in the air quantity delivered to the at least one cylinder from the feature correlating with the indicated mean pressure;
determining, in the second phase, a first value for a torque of the internal combustion engine from the feature correlating with the indicated mean pressure;
determining, in the second phase, a second value for the torque of the internal combustion engine based on a measured air quantity; and
comparing, in the second phase, the first value and the second value for the torque of the internal combustion engine to each other and providing a comparison result, and determining an error in the delivered air quantity of the at least one cylinder based on the comparison result.

17. The method of claim 16, wherein the second phase occurs at a defined steady-state operating point of the internal combustion engine.

18. The method of claim 16, wherein in the second phase a value for the air quantity error is determined and/or a value for the fuel quantity error is determined.

19. The method of claim 18, wherein a current value for the air quantity error is determined, by the value determined in the second phase for the fuel quantity error, when the internal combustion engine is not being operated in the second phase.

20. The method of claim 18, wherein the error in the delivered air quantity or the error in the delivered fuel quantity being respectively corrected, by the respective determined current value for the air quantity error or the value, determined in the second phase, for the fuel quantity error, when the internal combustion engine is not being operated in the second phase.

21. The method of claim 16, wherein the feature correlating with the indicated mean pressure is the indicated mean pressure of the at least one cylinder of the internal combustion engine.

22. The method of claim 16, wherein the feature correlating with the indicated mean pressure is a feature, based on a rotation speed, for the mechanical work of the at least one cylinder of the internal combustion engine.

23. The method of claim 16, wherein the ascertained error in the air quantity delivered to the at least one cylinder is used to correct an ascertainment of the air quantity.

24. The method of claim 16, wherein in the first phase:
the internal combustion engine is fueled in a lean mode,
a smoothness signal is evaluated, and an individual-cylinder feature of the at least one cylinder is determined,
an error in the fuel quantity delivered to the at least one cylinder is determined from the individual-cylinder feature.

25. The method of claim 16, wherein in a first phase an error in the delivered fuel quantity of the at least one cylinder is determined from a relationship between a torque of the internal combustion engine and the delivered fuel quantity.

26. The method of claim 16, wherein a non-torque-effective post-injection taking place in the first phase.

27. A calculation unit for determining a quantitative error in a fuel quantity and air quantity delivered to at least one cylinder of an internal combustion engine, comprising:
a processing arrangement configured to perform the following:
providing, in a first phase, a cylinder equalization with regard to the fuel quantity delivered to the internal combustion engine;
determining, in the first phase, an error in the fuel quantity delivered to the at least one cylinder therefrom;
operating, in a second phase, the internal combustion engine with a stoichiometric ratio of air quantity and fuel quantity;
sensing, in the second phase, a feature of the at least one cylinder correlating with an indicated mean pressure;
determining, in the second phase, an error in the air quantity delivered to the at least one cylinder from the feature correlating with the indicated mean pressure;
determining, in the second phase, a first value for a torque of the internal combustion engine from the feature correlating with the indicated mean pressure;
determining, in the second phase, a second value for the torque of the internal combustion engine based on a measured air quantity; and
comparing, in the second phase, the first value and the second value for the torque of the internal combustion engine to each other and providing a comparison result, and determining an error in the delivered air quantity of the at least one cylinder based on the comparison result.

28. A computer readable medium having a computer program, which is executable by a processor, comprising:
a program code arrangement having program code for determining a quantitative error in a fuel quantity and air quantity delivered to at least one cylinder of an internal combustion engine, by performing the following:
providing, in a first phase, a cylinder equalization with regard to the fuel quantity delivered to the internal combustion engine;
determining, in the first phase, an error in the fuel quantity delivered to the at least one cylinder therefrom;
operating, in a second phase, the internal combustion engine with a stoichiometric ratio of air quantity and fuel quantity;
sensing, in the second phase, a feature of the at least one cylinder correlating with an indicated mean pressure;
determining, in the second phase, an error in the air quantity delivered to the at least one cylinder from the feature correlating with the indicated mean pressure;
determining, in the second phase, a first value for a torque of the internal combustion engine from the feature correlating with the indicated mean pressure;
determining, in the second phase, a second value for the torque of the internal combustion engine based on a measured air quantity; and
comparing, in the second phase, the first value and the second value for the torque of the internal combustion engine to each other and providing a comparison result, and determining an error in the delivered air quantity of the at least one cylinder based on the comparison result.

29. The computer readable medium of claim 28, wherein the second phase occurs at a defined steady-state operating point of the internal combustion engine.
